(19) United States
(12) Patent Application Publication
(10) Pub. No.: US 2018/0195968 A1
(43) Pub. Date: Jul. 12, 2018

(54) INDICATING PENETRATION OF NON-AQUEOUS SOLVENT

(71) Applicant: Troy Corporation, Florha Park, NJ (US)

(72) Inventors: Geoffrey Vern Webb, Quakertown, PA (US); Gabrielle R. Watermen, Closter, NJ (US)

(21) Appl. No.: 15/863,598

(22) Filed: Jan. 5, 2018

Related U.S. Application Data
(60) Provisional application No. 62/444,596, filed on Jan. 10, 2017.

(51) Int. Cl. G01N 21/78; G01N 33/46; G01N 21/84

(52) U.S. Cl. CPC G01N 21/78; G01N 33/46; G01N 2021/8466; G01N 2021/8472; G01N 21/84

(57) ABSTRACT

A penetration indicator composition has been discovered which is soluble in solvent-based wood treating solutions, normally invisible to the human eye, and capable of exhibiting a visible color under test conditions. The indicator composition includes a hydrophobic borolane or a hydrophobic borate ester which exhibits a visually detectable color under the influence of a color change reagent.

The new indicator composition is chemically stabile under wood treating conditions and detectable at relatively low ranges of concentration. Oil-soluble preservatives such as pentachlorophenol, iodopropynyl butylcarbamate, propiconazole, and tebuconazole are compatible with the new indicator composition. Methods for indicating penetration of a non-aqueous solvent composition in wood or a wood-composite are also disclosed.

### BACKGROUND OF THE INVENTION

### Field of the Invention

The present invention relates to a penetration indicator and a process for determining the penetration depth of a non-aqueous solvent composition in wood and wood products.

### Description of the Related Art

Several processes for increasing the useful life of wood products by spraying liquid treating solutions on the wood or by immersing the wood in liquid treating solutions are known. The treating solutions may be either aqueous or non-aqueous liquids. Pressure-vacuum cycles are commonly utilized to force the treating solutions into wood and to remove air or the treating solutions from the wood. Manufacturers of wood products use these processes to increase the wood's resistance to weathering, microbial attack, and combustion, for example.

It is important to confirm the depth to which liquid treating solutions penetrate into wood or wood products. The sapwood of trees is relatively susceptible to decay, as compared to the heartwood. Unless sapwood is entirely impregnated with preservatives, decay can be expected to occur.

In some cases, the treating solutions are readily visible to the human eye even after drying and the depth of penetration is readily apparent in core-samples or cross-sectional samples of the treated wood. Creosote is an example of a wood treating solution that is visible in wood after drying.

In other cases, a colored treating agent or a colored dye is carried into the wood by the treating solution and can be detected after drying by visually inspecting cross-sectional samples of the treated wood. Of course, some consumers may find wood products that are permanently discolored by these colored agents or dyes undesirable.

When no solvent, treating agent, or dye is present and visible to the human eye; a color change indicator may be employed to enhance the visibility of a colorless or faintly colored indicator that is carried into the wood by the treating solution. For example, water-soluble boron compounds (such as boric acid, borax, and disodium octaborate tetrahydrate) are commonly used as penetration indicators in aqueous wood treating solutions.

A color change penetration indicator specifically tailored for enhancing the visibility of disodium octaborate tetrahydrate in wood is commercially available under the tradename Bora-Care Indicator Solution from Nisus Corporation of Rockford, Tenn., United States of America. Although soluble in water, disodium octaborate tetrahydrate exhibits relatively low solubility in non-aqueous solvents.

The American Wood Protection Association's AWPA Standard A78-12 entitled “Standard Method To Determine The Penetration Of Boron Containing Preservatives And Fire Retardants” describes a qualitative method for determining the penetration depth of boron containing preservatives and fire retardants in treated wood. The Standard describes a method which sequentially employs two color change reagents which are employed sequentially, the first reagent to produce a yellow color (sometimes described as a yellowish color) and the second reagent to produce a red color (sometimes described as a reddish color or a magenta color). The method described in the Standard offers no guidance regarding the composition of the boron containing preservatives or the means by which they are made to penetrate into wood.

Aqueous wood treating solutions are not appropriate for all wood treating applications. For example, some very desirable treating agents cannot be effectively dissolved or dispersed in aqueous solutions. Also, aqueous solutions tend to make wood swell and can affect the dimensional stability of the wood products.

U.S. Pat. No. 6,911,473 B2, which lists Raczek and Wetzel as inventors, describes a wood preservative composition comprising an organic preservative acid and a UV-active indicator substance for preserving wood and for detection of the sufficient preservation of wood. The '473 patent reports that a wood treated with this agent can be irradiated with a UV lamp to stimulate fluorescence that indicates the presence of the wood preservative.

U.S. Pat. No. 7,816,343 B2, which lists Mark C. Hoffman as inventor, describes a wood preservative composition which is soluble in a non-aqueous solvent and provides improved resistance to insect attack. The wood preservative composition reportedly contains a combination of fungicides, including a boron-containing compound, an organo iodine compound or compounds, and a triazole compound; an insecticide, such as a synthetic pyrethroid; and an organic solvent or carrier. The Hoffman patent defines the term “boron-containing fungicide” to include fungicides containing at least one boron compound, such as boric acid esters, which are soluble in organic solvents and have sterically hindered di-alcohol and tri-alcohol groups, but do not contain an anhydride bond between the boron atoms, including, but not limited to, trihexylene glycol biborate, trioctylene glycol biborate, and triisopropanolamine borate.

A need exists for a new indicator compositions and methods for indicating penetration of a non-aqueous solvent composition into wood or a wood-composite. The new indicator composition should be chemically stabile under wood treating conditions and detectable at relatively low ranges of concentration. Ideally, the new indicator will be soluble in solvent-based wood treating solutions, normally invisible to the human eye, and capable of exhibiting a visible color under test conditions.

### SUMMARY OF THE INVENTION

The inventor has discovered compositions and methods for indicating penetration depth of a non-aqueous solvent composition which carries a hydrophobic boron containing compound into wood or a wood-composite. Preservatives or other treating agents may be dissolved in and carried by the non-aqueous solvent composition, as well.

In one aspect, the invention is a method for indicating penetration of a non-aqueous solvent composition in wood or a wood composition. The wood or the wood composition is soaked with a non-aqueous solvent composition which includes a non-aqueous solvent and about 0.01 weight percent to about 5.0 weight percent of a boron containing compound dissolved in the non-aqueous solvent. The boron containing compound is hydrophobic and selected from the group consisting of borolanes, borate esters, and mixtures thereof. As the non-aqueous solvent enters the wood or the wood composite, the boron containing compound moves with the solvent.

The method includes applying a first reagent solution which contains curcumin and a first carrier liquid to a surface of the wood to produce a colored portion that exhibits a yellow color; applying a second reagent which includes an acid to the colored portion; and determining whether the colored portion exhibits a change in color from yellow to red. A change in color from yellow to red in the colored portion indicates that the boron-containing compound has penetrated the wood or the wood-composite to the red portion of the surface.

In another aspect the invention is an indicator composition for indicating penetration of a non-aqueous solvent preservative in wood or a wood-composite. The indicator composition consists essentially of a non-aqueous solvent and about 0.01 weight percent to about 5.0 weight percent of a boron-containing compound dissolved in the non-aqueous solvent. The boron-containing compound is selected from the group consisting of borolanes, borate esters, and mixtures thereof.

### DETAILED DESCRIPTION OF PREFERRED ASPECTS OF THE INVENTION

In a preferred aspect, the invention is a method for indicating penetration of a non-aqueous solvent composition in wood or a wood composition. The non-aqueous solvent composition of the invention includes a non-aqueous solvent and about 0.01 weight percent to about 5.0 weight percent of a hydrophobic boron containing compound dissolved in the non-aqueous solvent.

The boron-containing compound is selected from the group consisting of borolanes, borate esters, and mixtures thereof. The inventors have found that borolanes and borate esters are sufficiently stabile for industrial use and qualitatively detectable by their interaction with certain color change reagents.

Preferably, the boron-containing compound is selected from the group of chemically stabile, hydrocarbon soluble boron-containing compounds consisting of 2-isopropoxy-4,4,5,5-tetramethyl-[1,3,2]dioxaborolane; tris-2-ethylhexyl borate; tributyleneglycol biborate; trihexyleneglycol biborate; tri-n-butyl borate; trimethyl borate; and mixtures thereof. Most preferably, the boron-containing compound is selected from the group of highly detectable compounds consisting of 2-isopropoxy-4,4,5,5-tetramethyl-[1,3,2]dioxaborolane; tri-n-butyl borate; trimethyl borate; and mixtures thereof.

The non-aqueous solvent can be any essentially waterless solvent that is capable of dissolving about 0.01 weight percent to about 5.0 weight percent of the boron containing compound and will not hydrolyze a significant proportion of the boron containing compound. Preferably, the non-aqueous solvent is one that is presently in use in a conventional wood treating process.

For the present purposes, “Polarity Index” means the Snyder polarity index described in a technical paper by L. R. Snyder entitled “Classification of the Solvent Properties of Common Liquids” J. Chromatogr. Sci. (1978) 16 (6) pp 223-234. Preferably, the non-aqueous solvent of the invention exhibits a Polarity Index less than that of methanol (which exhibits a Polarity Index of 6.6), and more preferably less than that of ethanol (which exhibits a Polarity Index of 5.2).

Previously known solvent compositions employed in the wood protection industry include aliphatic mineral spirits. ShellSol D60™ (CAS Registry No. 6472-48-9), which is commercially available from Shell chemicals, is an example of aliphatic mineral spirits that are suitable for use as the non-aqueous solvent of the present invention, ShellSol D60™ reportedly consists predominantly of C10-C12 paraffins and naphthenes.

As another example of a suitable non-aqueous solvent, EXXSOL™ D60 Fluid (CAS Registry No. 64742-47-48) is commercially available from Exxon Mobil and reportedly contains hydrotreated light distillates of petroleum. Petroleum distillates containing less than 5000 ppm aromatics are especially suitable for use for use as the non-aqueous solvent of the present invention.

In the method, wood or a wood-composite is soaked with the non-aqueous solvent. For the present purposes “soaking” means thoroughly wetting with liquid or covering with liquid. Soaking may be accomplished, for example, by painting or spraying the liquid on wood or by immersing wood in the liquid. Wood treating processes that are conducted under atmospheric or positive pressure, or wood treating processes that include one or more sequential cycles of vacuum and positive pressure, can be successfully utilized in the present invention. Processes that include cycles of vacuum and positive pressure are preferred.

Wood treating processes conducted under positive pressure typically include immersing the wood in a non-aqueous solvent under positive pressure of about 50 to about 150 psig for about 10 to about 30 minutes at a temperature in the range of ambient to about 200 degrees F. Wood treating processes that include one or more sequential cycles of vacuum and positive pressure typically subject the wood to a vacuum of about 100 mm Hg for about 15 minutes to remove air from the wood, a positive pressure of about 50 to about 150 psig for about 10 to about 30 minutes at a temperature in the range of ambient to about 200 degrees F. while the wood is submerged in the non-aqueous solvent, and another vacuum of about 100 mm Hg for about 15 minutes to remove solvent from the wood.

For the present purposes, “yellow color” means any shade of yellow or yellowish color and “red color” means any shade of red, reddish or magenta color.

The following examples are intended to better communicate the invention and are not intended to limit the invention in any way.

### Example 1—Preparing Solutions of Hydrophobic Boron-Compound Solutes in Non-Aqueous Solvents for Use as Wood Penetration Indicators

Composition information for boron compound indicators No. 1-6 is presented below in Table 1. Each of the indicators was blended into mineral spirits to produce treating solutions that contained 0 (zero) weight percent. 0.01 weight percent, 0.10, weight percent, 0.25 percent, or 0.50 weight percent of the indicator. The blends with 0 weight percent of the respective indicator were used as blank solutions, for control purposes. Each of the blends exhibited the appearance of a clear solution.

TABLE 1

| Indicator | | | Abbreviated |
|---|---|---|---|
| No. | Chemical Name | CAS# | Name |
| 1 | 2-Isopropoxy-4,4,5,5- | 61676-62-8 | IPTMDOB |
| | tetramethyl- | | |
| | [1,3,2]dioxaborolane | | |
| 2 | Tris-2-ethylhexyl Borate | 2467-13-2 | 2EHB |
| 3 | Trimethyl borate | 121-43-7 | TMB |
| 4 | Tributyleneglycol biborate | 2665-13-6 | TBGBB |
| 5 | Trihexyleneglycol biborate | 100-89-0 | THGBB |
| 6 | Tri-n-butyl borate | 688-74-4 | TnBB |

Example 1 demonstrates that hydrophobic boron compounds, such as TnBB and TMBX, can be dissolved in a non-aqueous solvent, such as mineral spirits, to produce a clear solution.

### Example 2.—Treating Wood with Solutions of Hydrophobic Boron-Compounds Solutes in Non-Aqueous Solvents

Each of the treating solutions described above was employed in a pressure treating process to treat wood blocks. In addition, mineral spirits which contained essentially none of Indicator No. 1-6 were employed as blank treating solutions in the pressure treating process to treat wood blocks, for control purposes.

The blocks were composed of Southern Yellow Pine and shaped as cubes measuring 0.75 inches in each dimension. Prior to treating, the blocks were maintained at a temperature between 20° and 30° C.

For treatment, a desiccator provided with a separatory funnel or an auxiliary flask for holding excess treating solution and a vacuum gage were utilized. The blocks were submerged in the treating solution and then exposed to a vacuum of 100 mm mercury for 30 minutes, followed by 100 psig of positive pressure for 60 minutes pressure, and finally permitted to rest at atmospheric pressure for at least 30 minutes.

The blocks were then allowed to dry at ambient conditions. When dry to the touch, the blocks were split longitudinally along the wood grain. A chisel was used, with minimal contact, in order to minimize potential cross contamination.

Example 2 demonstrates that hydrophobic boron compounds, such as Indicators No 1-6, when dissolved in a non-aqueous solvent, such as mineral spirits, can be inserted into wood by conventional pressure treating processes.

### Example 3. Determining Penetration into Wood by Color Change of Curcumin Reagent which Detects Boron from Treating Solutions of Hydrophobic Boron-Compound Solute in Non-Aqueous Solvent

Wood blocks prepared as described above in Example 2 to produce test blocks for penetration testing in accordance with American Wood Protection Association (AWPA) standard A78-12.

Specifically, newly exposed surfaces of the test blocks were sprayed with Solution One of standard A78-12, allowed to dry, and then sprayed with Solution Two of standard A78-12. As set forth in standard A78-12, Solution One consists of 0.60 grams of curcumin dissolved in 500 mL of ethyl alcohol. Solution Two consists of 30 grams of salicylic acid dissolved in 100 mL of concentrated hydrochloric acid subsequently diluted to 500 mL with ethyl alcohol.

Surfaces of the test blocks which had been exposed to one of the Hydrophobic Boron-Compound containing treating solutions and colored yellow by Solution One turned a red color upon application of Solution two. In each case, the red color appeared to be coextensive with the depth of penetration of the boron-containing treating solutions into the wood block.

Surfaces of the test blocks which had been exposed to one of the blank treating solutions and colored yellow by Solution One did not turn red upon application of Solution Two.

The results of visual inspection after application of Solution Two are presented below in Table 2. The Chemical name for each Indicator is set forth above in Table 1.

TABLE 2
Indication by Color Change Reagent composed of Curcumin in
Ethanol, followed by Salicylic Acid and Hydrochloric Acid

| | Concentration of Indicator in Solvent (wt %) | | | | |
|---|---|---|---|---|---|
| Indicator | 0% (control) | 0.01% | 0.10% | 0.25% | 0.50% |
| IPTMDOB | Negative | Positive | Positive | Positive | Positive |
| 2EHB | Negative | Positive | Positive | Positive | Positive |
| TMB | Negative | Negative | Positive | Positive | Positive |
| TBGBB | Negative | Positive | Positive | Positive | Positive |
| THGBB | Negative | Positive | Positive | Positive | Positive |
| TnBB | Negative | Negative | Positive | Positive | Positive |

The data in Table 2 demonstrates that hydrophobic boron compounds; such as IPTMDOB, 2EHB, TMB, TBGBB, THGBB, and TnBB; can be dissolved in mineral spirits and that the penetration depth of the boron-containing mineral spirits into wood can be determined by a two-step application of curcumin in ethanol, followed by salicylic acid in hydrochloric acid.

### Example 4.—Preparing Solutions of Hydrophobic Boron-Compound Solutes in Non-Aqueous Solvents

Tri-n-butyl borate (also known as TnBB or CAS #688-74-4) was blended into mineral spirts to produce treating solutions that contained 0.3 weight percent TnBB, 1.0 weight percent TnBB, 3.0 weight percent TnBB, and 5.0 weight percent TnBB, respectively.

Trimethoxyboroxine (also known as TMBX or CAS #102-24-9) was blended into mineral spirts to produce a non-aqueous treating solution that contained 0.5 weight percent TMBX.

In each case, blending TnBB or TMBX into mineral spirits in the above described concentrations produced a clear solution.

Example 4 demonstrates that hydrophobic boron compounds, such as TnBB and TMBX, can be dissolved in a non-aqueous solvent, such as mineral spirits.

### Example 5.—Treating Wood with Solutions of Hydrophobic Boron-Compounds Solutes in Non-Aqueous Solvents

Each of the five treating solutions described above was employed in a Pressure Treating Process to treat five wood blocks. In addition, mineral spirits which contained essentially none of either of the borate compounds were employed as a blank treating solution in the Pressure Treating Process to treat five wood blocks for control purposes.

In each case, the Pressure Treating Process was conducted in a laboratory in a Laboratory Impregnation of Apparatus substantially as described in American Wood Protection Association Standard (AWPA) standard E10-16, Section 3.7. More specifically, mineral spirits in the amount of 300 grams were charged into an eight ounce jar equipped with a magnetic stirrer. A boron solution of known composition or a blank sample composed entirely of mineral spirits, was slowly added to the mineral spirits in the jar. With stirring for 10 minutes, the boron solution or the blank sample dissolved in the mineral spirits to produce a clear liquid treating solution.

Three at time, wooden blocks in the shape of 12 mm cubes were placed in a glass beaker located inside a Laboratory Impregnation Apparatus. The blocks were weighed down with a watch glass to prevent flotation.

The Impregnation Apparatus was closed and evacuated for 20 minutes to remove air from the blocks. Then treating solution was added to the impregnation apparatus over a period of 5 or more minutes to submerge the blocks. The submerged blocks were subjected a vacuum of 100 mm Hg for 30 minutes.

Pressure in the Impregnation Apparatus was increased to equal that of the ambient atmosphere and the blocks were. The blocks were wiped with a cloth to dry them.

Example 5 demonstrates that hydrophobic boron compounds, such as TnBB and TMBX, when dissolved in a non-aqueous solvent, such as mineral spirits, can be inserted into wood by conventional pressure treating processes.

### Example 6—Determining Penetration Depth into Wood of Solutions of Hydrophobic Boron-Compounds Solutes in Non-Aqueous Solvents Using Curcumin and Acids

Each of the 30 treated blocks described above in Preparative Example 4 was dried and split approximately in half to produce 60 test blocks for penetration testing in accordance with American Wood Protection Association (AWPA) standard A78-12.

Specifically, newly exposed surfaces of the test blocks were sprayed with Solution One of standard A78-12, allowed to dry, and then sprayed with Solution Two of standard A78-12. As set forth in standard A78-12, Solution One consists of 0.60 grams of curcumin dissolved in 500 mL of ethyl alcohol. Solution Two consists of 30 grams of salicylic acid dissolved in 100 mL of concentrated hydrochloric acid and then diluted to 500 mL with ethyl alcohol.

After application of Solution Two, surfaces of the test blocks which had been exposed to one of the boron containing treating solutions and subsequently colored yellow by Solution One turned a red color. In each case, the red color appeared to be coextensive with the depth of penetration of the boron-containing treating solutions into the wood block.

After application of Solution Two, surfaces of the test blocks which had been exposed to one of the blank treating solutions and subsequently colored yellow by Solution One exhibited no further color change. The results of visual inspection after application of Solution Two are presented below in Table 3. The Chemical name for each Indicator is set forth above in Table 1.

TABLE 3

| MS | TnBB | TnBB | TnBB | TnBB | TMBX |
|---|---|---|---|---|---|
| (control) | in MS | in MS | in MS | in MS | in MS |
| 0.0% | 0.5% | 1.0% | 3.0% | 5.0% | 0.5% |
| Negative | Positive | Positive | Positive | Positive | Positive |

The data in Table 3 demonstrates that hydrophobic boron compounds, such as TnBB and TMBX, can be dissolved in mineral spirits and that the penetration depth of the boron-containing mineral spirits into wood can be determined by a two-step application of curcumin in ethanol, followed by salicylic acid in hydrochloric acid.

### Example 7—Determining Penetration Depth into Wood of Solutions of Hydrophobic Boron-Compounds Solutes in Non-Aqueous Solvents Using Tumeric in Methanol

Each of 30 treated blocks treated with described above in Preparative Example 4 was dried and split approximately in half to produce 60 test blocks for penetration testing in accordance with American Wood Protection Association (AWPA) standard A78-12.

Specifically, newly exposed surfaces of the test blocks were sprayed with Solution One of standard A78-12, allowed to dry, and then sprayed with Solution Two of standard A78-12. As set forth in standard A78-12, Solution One consists of 0.60 grams of curcumin dissolved in 500 mL of ethyl alcohol. Solution Two consists of 30 grams of salicylic acid dissolved in 100 mL of concentrated hydrochloric acid and then diluted to 500 mL with ethyl alcohol.

Surfaces of the test blocks which had been exposed to one of the boron containing treating solutions and subsequently colored yellow by Solution One turned a red color upon application of Solution Two. In each case, the red color appeared to be coextensive with the depth of penetration of the boron-containing treating solutions into the wood block.

In contrast, surfaces of the test blocks which had been exposed to one of the blank treating solutions and subsequently colored yellow by Solution One exhibited no further color change upon application of Solution Two. The results of visual inspection after application of Solution Two are presented below in Table 4, in which TnBB means Tri-n-butyl borate and TMBX means Trimethoxyboroxine.

TABLE 4

| MS | TnBB | TnBB | TnBB | TnBB | TMBX |
|---|---|---|---|---|---|
| (control) | in MS | in MS | in MS | in MS | in MS |
| 0.0% | 0.5% | 1.0% | 3.0% | 5.0% | 0.5% |
| Negative | Positive | Positive | Positive | Positive | Positive |

Legend: “Positive” means a red color was observed
“Negative” means no red color was observed

The data in Table 6 demonstrates that hydrophobic boron compounds, such as TnBB and TMBX, can be dissolved in mineral spirits and that the penetration depth of the boron-containing mineral spirits into wood can be determined by means application of a two-part color change reagent, of the type described in WPA standard A78-12.

### Comparative Example 8.—Wood Penetration Depth of Hydrophobic Born-Containing Compounds not Indicated by Commercially Available Color Change Reagent

Treated blocks which had been treated as described above in Example 2 with hydrophobic boron-containing Indicators No. 1-5 were dried and split approximately in half to produce ten test blocks per Indicator for penetration testing by means of a commercially available indicator test solution sold under the trade name “Bora-Care Indicator Solution” by Nisus Corporation of Rockford, Tenn., United States of America. The indicator test solution is reportedly comprised of pyrocatechol violet in water with preservative. The indicator test solution is widely used for detecting the presence of boron in wood which has been treated with a water-soluble boron compound known as disodium octaborate tetrahydrate.

The newly exposed surfaces of the test blocks were sprayed with the indicator test solution and subsequently examined for a change in color. The results are presented below in Table 4. The Chemical name for each Indicator is set forth above in Table 1. No color change was observed in any of the test blocks

TABLE 4

| | | Concentration of Indicator in Solvent (wt %) | | | | |
|---|---|---|---|---|---|---|
| Indicator | Abbreviated | 0 wt % | | | | |
| No. | Name | (control) | 0.01 wt % | 0.10 wt % | 0.25 wt % | 0.50 wt % |
| 1 | IPTMDOB | Negative | Negative | Negative | Negative | Negative |
| 2 | 2EHB | Negative | Negative | Negative | Negative | Negative |
| 3 | TMB | Negative | Negative | Negative | Negative | Negative |
| 4 | TBGBB | Negative | Negative | Negative | Negative | Negative |
| 5 | THGBB | Negative | Negative | Negative | Negative | Negative |

Legend:
“Negative” means no red color was observed

The results of Comparative Example 7 indicate that a commercially available color change indicator test solution that is reportedly effective for indicating the presence of boron in wood which has been treated with a water-soluble boron compound is not effective for indicating the presence of boron in wood which has been treated with a hydrophobic boron-containing compound

The above Examples do not limit the invention in any way. The invention is defined solely by the appended claims. For the present purposes, “consists essentially of” means that the scope of an associated patent claim is limited to the specified materials or steps and those that do not materially affect the basic and novel characteristic(s) of the claimed invention.

What is claimed is:

1. A method for indicating penetration of a non-aqueous solvent composition in wood or a wood-composite, which method comprises:
providing wood or a wood-composite which has been soaked with a non-aqueous solvent composition that includes
a non-aqueous solvent, and
about 0.01 weight percent to about 5.0 weight percent of a hydrophobic boron-containing compound dissolved in the non-aqueous solvent, which boron-containing compound is selected from the group consisting of borolanes, borate esters, and mixtures thereof; and
applying a first reagent solution which includes curcumin on a surface of the wood or the wood-composite to produce a yellow portion;
applying a second reagent solution which includes an acid to the yellowish portion; and
determining whether the yellowish portion exhibits a change in color from yellow to red;
whereby a change in color from yellow to red in the colored portion indicates that the boron-containing compound has penetrated the wood or the wood-composite to the red portion.

2. The method of claim 1, in which
the first reagent solution includes a non-aqueous solvent, and
the second reagent solution includes salicylic acid and hydrochloric acid.

3. The method of claim 1, in which the Polarity Index of the non-aqueous solvent is 6.6 or less.

4. The method of claim 1, in which the Polarity Index of the non-aqueous solvent is less than 5.2 or less.

5. The method of claim 1, in which the non-aqueous solvent is a petroleum distillate.

6. The method of claim 5, in which less than 5000 ppm aromatics are present in the petroleum distillate.

7. The method of claim 1, in which the boron-containing compound is selected from the group consisting of 2-isopropoxy-4,4,5,5-tetramethyl-[1,3,2]dioxaborolane; tris-2-ethylhexyl borate; tributyleneglycol biborate; trihexyleneglycol biborate; tri-n-butyl borate; trimethyl borate; and mixtures thereof.

8. The method of claim 7, in which the boron-containing compound is selected from the group consisting of 2-isopropoxy-4,4,5,5-tetramethyl-[1,3,2]dioxaborolane; tri-n-butyl borate; trimethyl borate; and mixtures thereof.

9. The method of claim 2 in which the non-aqueous solvent includes a preservative.

10. The method of claim 9 in which the preservative is selected from the group consisting of pentachlorophenol, iodopropynyl butylcarbamate, propiconazole, tebuconazole, and mixtures thereof.

11. A method for indicating penetration of a non-aqueous solvent composition in wood or a wood-composite, which method comprises:
soaking wood or a wood-composite with a non-aqueous solvent composition that includes
a non-aqueous solvent, and
about 0.01 weight percent to about 5.0 weight percent of a hydrophobic boron-containing compound dissolved in the non-aqueous solvent, which boron-containing compound is selected from the group consisting of borolanes, borate esters, and mixtures thereof and
applying a first reagent solution which includes curcumin a surface of the wood or the wood-composite to produce a yellow portion;
applying a second reagent solution which includes an acid to the yellowish portion; and
determining whether the yellowish portion exhibits a change in color from yellow to red;
whereby a change in color from yellow to red in the colored portion indicates that the boron-containing compound has penetrated the wood or the wood-composite to the red portion.

12. The method of claim 11, in which the Polarity Index of the non-aqueous solvent is 6.6 or less.

13. The method of claim 12, in which the Polarity Index of the non-aqueous solvent is less than 5.2 or less.

14. The method of claim 11, in which the non-aqueous solvent is a petroleum distillate.

15. The method of claim 14, in which less than 5000 ppm aromatics are present in the petroleum distillate.

16. The method of claim 11, in which the boron-containing compound is selected from the group consisting of 2-isopropoxy-4,4,5,5-tetramethyl-[1,3,2]dioxaborolane; tris-2-ethylhexyl borate; tributyleneglycol biborate; trihexyleneglycol biborate; tri-n-butyl borate; trimethyl borate; and mixtures thereof.

17. The method of claim 11, in which the boron-containing compound is selected from the group consisting of 2-isopropoxy-4,4,5,5-tetramethyl-[1,3,2]dioxaborolane; tri-n-butyl borate; trimethyl borate; and mixtures thereof.

18. The method of claim 11, in which soaking the wood or the wood-composite with the non-aqueous solvent includes spraying the non-aqueous solvent on the wood or the wood-composite, or immersing wood or the wood-composite in the non-aqueous solvent.

19. The method of claim 18, in which soaking the wood or the wood-composite with the non-aqueous solvent includes forcing the non-aqueous solvent into the wood or the wood-composite under pressure.

20. The method of claim 1, in which soaking the wood or the wood-composite to the non-aqueous solvent includes drawing air from the wood or wood-composite by establishing a partial vacuum in or near the wood or wood-composite.

21. The method of claim 1, in which the non-aqueous solvent includes a preservative.

22. The method of claim 1 in which the preservative is selected from the group consisting of pentachlorophenol, iodopropynyl butylcarbamate, propiconazole, tebuconazole, and mixtures thereof.

23. An indicator composition for indicating penetration of a non-aqueous solvent composition in wood or a wood-composite, which indicator consists essentially of:
a non-aqueous solvent, and
about 0.01 weight percent to about 5.0 weight percent of a hydrophobic boron-containing compound dissolved in the non-aqueous solvent;
which boron-containing compound is selected from the group consisting of borolanes, borate esters, and mixtures thereof.

24. The composition of claim 23, in which the Polarity Index of the non-aqueous solvent is less than 6.6 or less.

25. The composition of claim 23 in which the non-aqueous solvent is a petroleum distillate.

26. The composition of claim 25, in which less than 5,000 ppm of aromatic hydrocarbons are present in the petroleum distillate.

27. The composition of claim 23, in which the boron-containing compound is selected from the group consisting of 2-isopropoxy-4,4,5,5-tetramethyl-[1,3,2]dioxaborolane; tris-2-ethylhexyl borate; tributyleneglycol biborate; trihexyleneglycol biborate; tri-n-butyl borate; trimethyl borate; and mixtures thereof.

28. The composition of claim 23 in which the boron-containing compound is selected from the group consisting of 2-isopropoxy-4,4,5,5-tetramethyl-[1,3,2]dioxaborolane, tri-n-butyl borate; and trimethyl borate, and mixtures thereof.
